(19) United States
(12) Patent Application Publication
(10) Pub. No.: US 2025/0025697 A1
(43) Pub. Date: Jan. 23, 2025

(54) INFERRING LEAD PLACEMENT BASED ON SENSED BIOMARKERS

(71) Applicant: Boston Scientific Neuromodulation Coporation, Valencia, CA (US)

(72) Inventors: Max Witwer, Grand Junction, CO (US); Tianhe Zhang, Studio City, CA (US)

(21) Appl. No.: 18/774,049

(22) Filed: Jul. 16, 2024

Related U.S. Application Data
(60) Provisional application No. 63/527,220, filed on Jul. 17, 2023.

(51) Int. Cl. A61N 1/36; A61N 1/02; A61N 1/05; A61N 1/372

(52) U.S. Cl. CPC A61N 1/36135; A61N 1/025; A61N 1/0553; A61N 1/372

(57) ABSTRACT

A system may include an electrode arrangement and a neurostimulator. The electrode arrangement may have a length generally in a rostral-caudal direction and a width generally in a mediolateral direction. The electrode arrangement may include a first set and a second set of electrodes spaced across the width. The neurostimulator may be configured to stimulate a sequence of locations of the patient using subsets of the first set of electrodes, and for each stimulated location in the stimulated sequence of locations identify corresponding response measures for each one of the second set of electrodes. The physiological midline may be inferred or identified from these measurements, which may be used to guide lead placement and/or programming.

### CLAIM OF PRIORITY

This application claims the benefit of U.S. Provisional Application No. 63/527,220 filed on Jul. 17, 2023, which is hereby incorporated by reference in its entirety.

### TECHNICAL FIELD

This document relates generally to medical systems, and more particularly, but not by way of limitation, to systems, devices, and methods for determining a spinal cord physiological midline which may be useful for placing electrodes and may be useful for programming or reprogramming a stimulation therapy.

### BACKGROUND

Neural modulation has been proposed as a therapy for a number of conditions. Often, neural modulation and neural stimulation may be used interchangeably to describe excitatory stimulation that causes action potentials as well as inhibitory and other effects. Examples of neuromodulation include Spinal Cord Stimulation (SCS), Deep Brain Stimulation (DBS), Peripheral Nerve Stimulation (PNS), and Functional Electrical Stimulation (FES). SCS, by way of example and not limitation, has been used to treat chronic pain syndromes. SCS has conventionally been used as a pain therapy, but may be implemented for other therapies as well.

Some neural targets may be complex structures with different types of nerve fibers. An example of such a complex structure is the neuronal elements in and around the spinal cord targeted by SCS. Furthermore, the number of available electrodes combined with the ability to generate a variety of complex electrical waveforms (e.g., pulses), presents a huge selection of modulation parameter sets to the clinician or patient. For example, if the neuromodulation system to be programmed has sixteen electrodes, millions of modulation parameter sets may be available for programming into the neuromodulation system. SCS systems are implanted using a surgical procedure that places one or more leads near the spinal cord, and the neural stimulator is implanted under the skin. Although the SCS lead(s) have a plurality of electrodes that provide flexibility in programming the size, shape and location of the neuromodulation field, the lead(s) must still be appropriately positioned so that the neurostimulator can be programmed to deliver effective neuromodulation energy to the correct nerves for the desired therapy.

Dorsal column (DC) fibers within the spinal cord are somatotopically organized medially/laterally. such that mediolateral placement of electrode arrangements for SCS should be considered. For example, knowing where the physiological midline is relative to the electrode arrangement is useful for predicting activation and clinical effects and is useful for steering stimulation to the correct dermatome(s). Therefore, stimulation prediction algorithms, closed loop stimulation algorithms, and the like may use physiological midline information.

Some systems may use image-guided techniques, such as fluoroscopy, to guide placement of a lead representation on a user interface, which may then be used to program a neurostimulator to stimulate targets. However, the image guided techniques may introduce inaccuracies because the radiology technician can arbitrarily place and orientate the fluoroscope with respect to the patient. Therefore, the angle of imaging and the quality of the image is highly dependent on the angle that the radiology technician sets the fluoroscope.

### SUMMARY

Various embodiments of the present subject matter may be used to infer lead placement with respect to the physiological midline by stimulating the spinal cord at different points and then sensing at different points of the spinal cord to infer where the midline is you are and to either recommend a new lead placement or adjust stimulation or the expected stimulation based on the inferred midline.

An example (e.g., Example 1) of a system may include an electrode arrangement and a neurostimulator configured to be connected to the electrode arrangement. The electrode arrangement may be configured to be positioned near a spine of a patient. The electrode arrangement may have a length generally in a rostral-caudal direction of the patient and a width generally in a mediolateral direction patient. The electrode arrangement may include a first set of electrodes spaced across the width and a second set of electrodes spaced across the width. The neurostimulator may be configured to stimulate a sequence of locations of the patient using subsets of the first set of electrodes, and for each stimulated location in the stimulated sequence of locations identify corresponding response measures for each electrode subset within the second set of electrodes. The stimulated sequence of locations may include at least a first location and a second location and the second set of electrodes may include at least a first electrode subset and a second electrode subset. The corresponding response measures may include, for the first location, a first first-location response measure for the first electrode subset and a second first-location response measure for the second electrode subset. The corresponding response measures may include, for the second location, a first second-location response measure for the first electrode subset and a second second-location response measure for the second electrode subset. The system may be configured to identify a physiological midline based on the corresponding response measures for each stimulated location in the stimulated sequence of locations. The first and second “first-location” response measures may be recorded or measured simultaneously or may be recorded or measured at different times. The first and second “second-location” response measures also may be recorded or measured simultaneously or may be recorded or measured at different times. The electrode subsets within the second set of electrodes may include one electrode such as may be used for monopolar sensing or two (or more) electrodes such as may be used for differential sensing.

In Example 2, the subject matter of Example 1 may optionally be configured such that the neurostimulator is configured to identify the physiological midline.

In Example 3, the subject matter of any one or more of Examples 1-2 may optionally be configured such that the system includes a programmer configured to program the neurostimulator, and the programmer is configured to identify the physiological midline.

In Example 4, the subject matter of Example 3 may optionally be configured such that the programmer is configured to program the neurostimulator using the identified physiological midline.

In Example 5, the subject matter of any one or more of Examples 3-4 may optionally be configured such that the programmer is configured to compare changes in the corresponding response measures to detect relative movement between a spine and the electrode arrangement and reprogram a neurostimulator connected to the electrode arrangement based on the detected relative movement. The relative movement may be caused by lead migration and/or spinal movement such as may occur with activity or postural changes.

In Example 6, the subject matter of any one or more of Examples 3-5 may optionally be configured such that the programmer includes a display and is configured to provide on the display a plot of the corresponding response measures for each of the stimulated locations. The displayed plot may be used to identify the physiological midline.

In Example 7, the subject matter of any one or more of Examples 1-6 may optionally be configured such that the neurostimulator is configured to stimulate the sequence of locations by moving at least one pole for delivered stimulation energy to stimulate the sequence of locations. The pole(s) may be a single pole such as a cathodic or anodic pole. The pole(s) may include a bipole, a guarded tripole or other electrode geometries that can be moved to stimulate sequence of locations.

In Example 8, the subject matter of Example 7 may optionally be configured such that the at least one pole includes a bipole, the neurostimulator is configured to stimulate the sequence of locations by moving the bipole for the delivered stimulation energy to stimulate the sequence of locations, and the bipole includes a cathodic pole and an anodic pole.

In Example 9, the subject matter of Example 7 may optionally be configured such that the at least one pole includes a mediolateral guarded tripole, the neurostimulator is configured to stimulate the sequence of locations by using the mediolateral guarded tripole to stimulate at least some of the locations, and the mediolateral guarded tripole includes a cathodic pole generally between at least two anodic poles.

In Example 10, the subject matter of Example 7 may optionally be configured such that the at least one pole includes a monopole used to stimulate at least some of the locations.

In Example 11, the subject matter of Example 7 may optionally be configured such that the electrode arrangement includes at least a third set of electrodes spaced across the width. For each stimulated location in the stimulated sequence of locations, the neurostimulator may be configured to identify corresponding response measures for each electrode subset within the third set of electrodes and the system may be configured to identify the physiological midline based on the corresponding response measures for each electrode subset within the second set of electrodes and the corresponding response measures for each electrode subset within of the third set of electrodes. Or for each stimulated location in the stimulated sequence of locations, the neurostimulator may be configured to use corresponding electrodes within the second set of electrodes and the third set of electrodes to form corresponding differential pairs for sensing, and use the corresponding differential pairs to identify the corresponding response measures.

In Example 12, the subject matter of Example 11 may optionally be configured such that the programmer is configured to trend plots across rows for the second set of electrodes and the at least the third set of electrodes to infer lead alignment with the physiological midline.

In Example 13, the subject matter of Example 11 may optionally be configured such that the programmer is configured to trend plots along individual columns for electrodes in the second and the least the third sets of electrodes.

In Example 14, the subject matter of any one or more of Examples 1-13 may optionally be configured such that the electrode arrangement is on a paddle lead, and the electrode arrangement includes at least three columns of electrodes.

In Example 15, the subject matter of any one or more of Examples 1-14 may optionally be configured such that the corresponding response measures includes measures for at least one feature of an evoked compound action potential (ECAP) selected from a magnitude, and area under curve (AUC), a curve length (CL), a power-spectra property, a wavelet property, a correlation with a template, progression with a known parameter, or at least one latency between different channels.

Example 16 includes subject matter (such as a method, means for performing acts, machine readable medium including instructions that when performed by a machine cause the machine to perform acts, or an apparatus to perform). The subject matter may be performed using an electrode arrangement positioned near a spine of a patient, where the electrode arrangement has a length generally in a rostral-caudal direction of the patient and a width generally in a mediolateral direction patient. The electrode arrangement may include a first set of electrodes spaced across the width and a second set of electrodes spaced across the width. The method may include stimulating a sequence of locations of the patient using subsets of the first set of electrodes, and for each stimulated location in the stimulated sequence of locations, identifying corresponding response measure for each electrode subset within the second set of electrodes. The stimulated sequence of locations may include at least a first location and a second location and the second set of electrodes may include at least a first electrode subset and a second electrode subset. The corresponding response measures may include, for the first location, a first first-location response measure for the first electrode subset and a second first-location response measure for the second electrode subset. The corresponding response measures may include, for the second location, a first second-location response measure for the first electrode subset and a second second-location response measure for the second electrode subset. The method may further include identifying a physiological midline based on the corresponding response measures for each stimulated location in the stimulated sequence of locations. The first and second “first-location” response measures may be recorded or measured simultaneously or may be recorded or measured at different times. The first and second “second-location” response measures also may be recorded or measured simultaneously or may be recorded or measured at different times. The electrode subsets within the second set of electrodes may include one electrode such as may be used for monopolar sensing or two (or more) electrodes such as may be used for differential sensing.

In Example 17, the subject matter of Example 16 may optionally be configured such that the stimulating the sequence of locations includes moving at least one pole for delivered stimulation energy to stimulate the sequence of locations.

In Example 18, the subject matter of Example 17 may optionally be configured such that the pole(s) may include a bipole, the stimulating the sequence of locations includes moving the bipole for the delivered stimulation energy to stimulate the sequence of locations, and the bipole includes a cathodic pole and an anodic pole.

In Example 19, the subject matter of Example 17 may optionally be configured such that the pole(s) may include a mediolateral guarded tripole, the stimulating the sequence of locations includes using the mediolateral guarded tripole to stimulate at least some of the locations, and the mediolateral guarded tripole includes a cathodic pole generally between at least two anodic poles.

In Example 20, the subject matter of Example 17 may optionally be configured such that the pole(s) may include a monopole used to stimulate at least some of the locations.

In Example 21, the subject matter of Example 17 may optionally be configured such that the electrode arrangement includes at least a third set of electrodes spaced across the width. For each stimulated location in the stimulated sequence of locations, the identifying corresponding response measures may include identifying corresponding response measures for each electrode subset within the third set of electrodes, and the system may be configured to identify the physiological midline based on the corresponding response measures for each electrode subset within the second set of electrodes and the corresponding response measures for each electrode subset within of the third set of electrodes. Or for each stimulated location in the stimulated sequence of locations, the identifying corresponding response measures may include using corresponding electrodes within of the second set of electrodes and the third set of electrodes to form corresponding differential pairs for sensing, and use the corresponding differential pairs to identify the corresponding response measures.

In Example 22, the subject matter of Example 21 may optionally be configured to further include comparing trend plots across rows for the second set of electrodes and the at least the third set of electrodes to infer lead alignment with the physiological midline.

In Example 23, the subject matter of Example 21 may optionally be configured to further include comparing trend plots across individual columns for electrodes in the second and the least the third sets of electrodes.

In Example 24, the subject matter of any one or more of Examples 16-23 may optionally be configured such that the first set of electrodes is proximate a rostral end of the electrode arrangement and the second set of electrodes is proximate to a caudal end of the electrode arrangement.

In Example 25, the subject matter of any one or more of Examples 16-24 may optionally be configured such that the electrode arrangement is on a paddle lead.

In Example 26, the subject matter of any one or more of Examples 16-25 may optionally be configured such that the electrode arrangement includes at least three columns of electrodes.

In Example 27, the subject matter of any one or more of Examples 16-26 may optionally be configured such that the electrode arrangement includes rows of electrodes, and at least some of the electrodes in the rows of electrodes are staggered with respect to each other.

In Example 28, the subject matter of any one or more of Examples 16-27 may optionally be configured such that the corresponding response measures includes measures for at least one feature of an evoked compound action potential (ECAP) selected from a magnitude, an area under curve (AUC), a curve length (CL), a power-spectra property, a wavelet property, a correlation with a template, progression with a known parameter, or at least one latency between different channels.

In Example 29, the subject matter of any one or more of Examples 16-28 may optionally be configured to further include comprising programming a neurostimulator connected to the electrode arrangement to deliver neuromodulation using the identified physiological midline.

In Example 30, the subject matter of any one or more of Examples 16-29 may optionally be configured to further include comparing changes in the corresponding response measures to detect lead migration, and reprogramming a neurostimulator connected to the electrode arrangement based on the detected lead migration.

In Example 31, the subject matter of any one or more of Examples 16-30 may optionally be configured to further include displaying a plot of the corresponding response measures for each of the stimulated locations. The displayed plot may be used to identify the physiological midline.

Example 32 includes subject matter (such as a non-transitory machine-readable medium including instructions, which when executed by a machine, cause the machine to perform a method performed using an electrode arrangement positioned near a spine of a patient. The electrode arrangement may have a length generally in a rostral-caudal direction of the patient and a width generally in a mediolateral direction patient. The electrode arrangement may include a first set of electrodes spaced across the width and a second set of electrodes spaced across the width. The method performed using the machine may include stimulating a sequence of locations of the patient using subsets of the first set of electrodes, and for each stimulated location in the stimulated sequence of locations, identifying corresponding response measure for each electrode subset within the second set of electrodes. The stimulated sequence of locations may include at least a first location and a second location and the second set of electrodes may include at least a first electrode subset and a second electrode subset. The corresponding response measures may include, for the first location, a first first-location response measure for the first electrode subset and a second first-location response measure for the second electrode subset. The corresponding response measures may include, for the second location, a first second-location response measure for the first electrode subset and a second second-location response measure for the second electrode subset. The method may further include identifying a physiological midline based on the corresponding response measures for each stimulated location in the stimulated sequence of locations. The first and second “first-location” response measures may be recorded or measured simultaneously or may be recorded or measured at different times. The first and second “second-location” response measures may be recorded or measured simultaneously or may be recorded or measured at different times. The electrode subsets within second set of electrodes may include one electrode such as may be used for monopolar sensing or two (or more) electrodes such as may be used for differential sensing.

In further examples, the subject matter of Example 32 may be configured such that the method performed by the machine may include any of the subject matter recited in Examples 17-32.

For example, in Example 33, the subject matter of Example 32 may optionally be configured such that the method performed by the machine further includes identifying a physiological midline based on the corresponding response measures for each stimulated location in the stimulated sequence of locations.

For example, in Example 34, the subject matter of Example 32 may optionally be configured such that the electrode arrangement is on a paddle lead, and the electrode arrangement includes at least three columns of electrodes.

For example, in Example 35, the subject matter of Example 32 may optionally be configured such the corresponding response measures includes measures for at least one feature of an evoked compound action potential (ECAP) selected from a magnitude, a power-spectra property, a wavelet property, a correlation with a template, progression with a known parameter, or at least one latency between different channels.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, an embodiment of a neuromodulation system.

FIG. 2 illustrates an embodiment of a modulation device, such as may be implemented in the neuromodulation system of FIG. 1.

FIG. 3 illustrates an embodiment of a programming system such as a programming device, which may be implemented as the programming device in the neuromodulation system of FIG. 1.

FIG. 4 illustrates, by way of example, an embodiment of a SCS system, which also may be referred to as a Spinal Cord Modulation (SCM) system.

FIG. 5 illustrates, by way of example and not limitation, an example of an electrode arrangement with a stimulating set of electrodes and with recording sets of electrodes.

FIG. 6 illustrates, by way of example and not limitation, a method to identify a midline using responses to a stimulated sequence of locations.

FIG. 7 illustrates, by way of example and not limitation, exclusive subsets of electrodes such as may be used in the stimulating electrodes (e.g., first set in FIG. 5) or recording electrodes (e.g., second set in FIG. 6).

FIG. 8 illustrates, by way of example and not limitation, nonexclusive subsets of electrodes such as may be used in the stimulating electrodes (e.g., first set in FIG. 5) or recording electrodes (e.g., second set in FIG. 6).

FIG. 9 illustrates, by way of example and not limitation, recording responses using a second set of electrodes when a sequence of locations is stimulated using subsets within a first set of electrodes.

FIG. 10 illustrates, by way of example and not limitation, a paddle lead that shifts a target bipole mediolaterally while recording from other contacts.

FIG. 11 illustrates, by way of example and not limitation, a paddle lead that shifts a mediolateral guarded tripole mediolaterally while recording from other contacts.

FIG. 12 illustrates, by way of example and not limitation, a paddle lead that builds plots in a rostrocaudal direction.

FIG. 13 illustrates, by way of example and not limitation, mediolateral biomarker measurements.

FIG. 14 illustrates, by way of example and not limitation, a physiological midline identification and recommendation.

FIG. 15 illustrates, by way of example and not limitation, a change in feature based on mediolateral displacement from midline.

FIG. 16 illustrates, by way of example and not limitation, a feature response for left mediolateral paddle displacement.

FIG. 17 illustrates, by way of example and not limitation, a feature response for properly placed paddle.

FIG. 18 illustrates, by way of example and not limitation, a feature response for left mediolateral paddle displacement.

FIG. 19 illustrates, by way of example and not limitation, an effect of increasing amplitude of evoking stimulation.

FIG. 20 illustrates, by way of example and not limitation, stimulation configurations. In this non-limiting example used to model the response, the mediolateral stimulation sweep may move from a left bipole, to a left tripole, to an extended tripole, to a right tripole and finally to a right bipole.

FIG. 21 illustrates, by way of example and not limitation, left lead midline displacement evoking in response to 3 mA stimulation and average differentially sensing at two levels.

FIG. 22 illustrates, by way of example and not limitation, a paddle center on midline displacement evoking in response to 3 mA stimulation and average differentially sensing at two levels.

FIG. 23 illustrates, by way of example and not limitation, right lead midline displacement evoking midline displacement evoking in response to 3 mA stimulation and average differentially sensing at two levels.

FIG. 24 illustrates, by way of example and not limitation, a simulation of a rotation of a paddle lead.

FIG. 25 illustrates, by way of example and not limitation, an interpolation of a sensing contact based on a nearest grid point.

FIG. 26 illustrates, by way of example and not limitation, plots that illustrate that rotation of the paddle lead along the mediolateral-rostrocaudal access can be detected using three pairs of contacts on the same paddle lead.

FIG. 27 illustrates, by way of example and not limitation, rotation angles of a paddle lead with respect to the midline

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

The present subject matter may be used to determine absolute or relative offsets from physiological midline based on biomarker/electrophysiological measurements and to use those measurements for programming purposes. The measurements capture responses to stimulating a mediolateral sequence of locations along the spine. For at least some of these locations, the stimulation may cause action potentials along the dorsal column (DC) fibers which may be measured using other electrodes on the lead. The midline may be inferred or identified based on the mediolateral progression of biomarker signals. The mediolateral lead migration may be identified based on changes in the relative measurements between two or more mediolaterally offset contacts or leads. Further, dorsal ventral may also be identified in some embodiments. The system may be configured to recommend programming or reprogramming using this information.

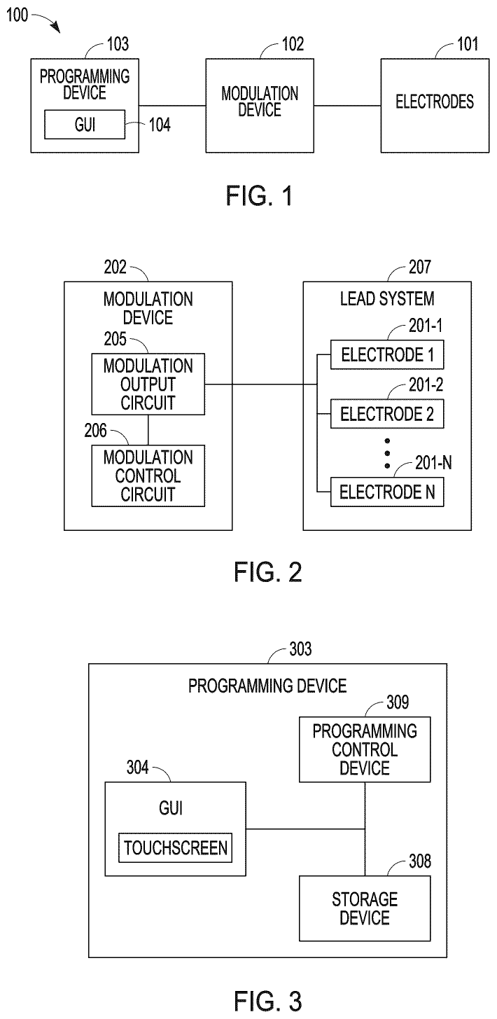

FIG. 1 illustrates, by way of example and not limitation, an embodiment of a neuromodulation system. The illustrated system 100 includes electrodes 101, a modulation device 102, and a programming system such as a programming device 103. The programming system may include multiple devices. The electrodes 101 are configured to be placed on or near one or more neural targets in a patient. The modulation device 102 is configured to be electrically connected to electrodes 101 and deliver neuromodulation energy, such as in the form of electrical pulses, to the one or more neural targets though electrodes 101. The delivery of the neuromodulation is controlled using a stimulation configuration. The stimulation configuration may include a modulation parameter set to specify the electrical waveform (e.g., pulses or pulse patterns or other waveform shapes). The stimulation configuration may also include an electrode configuration (e.g., selection of active electrodes through which the electrical waveform is delivered, the polarity of active electrodes, and the fractionalization to determine the energy distribution among the active electrodes. The modulation parameter set may also define the electrode configuration. The stimulation configuration may also include pulse timing information, such as a time delay and phase offset for a stimulation channel. In various embodiments, at least some parameters of the plurality of modulation parameters are programmable by a user, such as a physician or other caregiver. The programming device 103 provides the user with accessibility to the user-programmable parameters. In various embodiments, the programming device 103 is configured to be communicatively coupled to modulation device via a wired or wireless link. In various embodiments, the programming device 103 includes a graphical user interface (GUI) 104 that allows the user to set and/or adjust values of the user-programmable modulation parameters.

FIG. 2 illustrates an embodiment of a modulation device 202, such as may be implemented in the neuromodulation system 100 of FIG. 1. The illustrated embodiment of the modulation device 202 includes a modulation output circuit 205 and a modulation control circuit 206. Those of ordinary skill in the art will understand that the neuromodulation system may include additional components such as sensing circuitry for patient monitoring and/or feedback control of the therapy, telemetry circuitry and power. The modulation output circuit 205 produces and delivers the neuromodulation. Neuromodulation pulses are provided herein as an example. However, the present subject matter is not limited to pulses, but may include other electrical waveforms (e.g., waveforms with different waveform shapes, and waveforms with various pulse patterns). The modulation control circuit 206 controls the delivery of the neuromodulation pulses using the plurality of modulation parameters. The lead system 207 includes one or more leads each configured to be electrically connected to modulation device 202 and a plurality of electrodes 201-1 to 201-N distributed in an electrode arrangement using the one or more leads. Each lead may have an electrode array consisting of two or more electrodes, which also may be referred to as contacts. Multiple leads may provide multiple electrode arrays to provide the electrode arrangement. Each electrode is a single electrically conductive contact providing for an electrical interface between modulation output circuit 205 and tissue of the patient, where N≥2. The neuromodulation pulses are each delivered from the modulation output circuit 205 through a set of electrodes selected from the electrodes 201-1 to 201-N. The number of leads and the number of electrodes on each lead may depend on, for example, the distribution of target(s) of the neuromodulation and the need for controlling the distribution of electric field at each target. In one embodiment, by way of example and not limitation, the lead system includes two leads each having eight electrodes. Some embodiments may use a lead system that includes a paddle lead.

The actual number and shape of leads and electrodes may vary for the intended application. An implantable waveform generator may include an outer case for housing the electronic and other components. The outer case may be composed of an electrically conductive, biocompatible material, such as titanium, that forms a hermetically-sealed compartment wherein the internal electronics are protected from the body tissue and fluids. In some cases, the outer case may serve as an electrode (e.g., case electrode). The waveform generator may include electronic components, such as a controller/processor (e.g., a microcontroller), memory, a battery, telemetry circuitry, monitoring circuitry, modulation output circuitry, and other suitable components known to those skilled in the art. The microcontroller executes a suitable program stored in memory, for directing and controlling the neuromodulation performed by the waveform generator. A stimulation configuration determines how energy is delivered through the electrodes. A stimulation configuration may include a modulation parameter set which may include amplitude, pulse width, and frequency, an electrode configuration which may include fractionalization and polarity, and pulse timing which may include a time delay and phase offset. Electrical modulation energy is provided to the electrodes in accordance with a set of modulation parameters programmed into the pulse generator. By way of example but not limitation, the electrical modulation energy may be in the form of a pulsed electrical waveform. The stimulation configuration may comprise electrode combinations, which define the electrodes that are activated as anodes (positive), cathodes (negative), and turned off (zero), percentage of modulation energy assigned to each electrode (fractionalized electrode configurations), and electrical pulse parameters, which define the pulse amplitude (measured in milliamps or volts depending on whether the pulse generator supplies constant current or constant voltage to the electrode array), pulse width (measured in microseconds), pulse rate (measured in pulses per second), and burst rate (measured as the modulation on duration X and modulation off duration Y). Electrodes that are selected to transmit or receive electrical energy are referred to herein as “activated,” while electrodes that are not selected to transmit or receive electrical energy are referred to herein as “non-activated.”

Electrical modulation occurs between or among a plurality of activated electrodes, one of which may be the case of the waveform generator. The system may be capable of transmitting modulation energy to the tissue in a monopolar or multipolar (e.g., bipolar, tripolar, etc.) fashion. Monopolar modulation occurs when a selected one of the lead electrodes is activated along with the case of the waveform generator, so that modulation energy is transmitted between the selected electrode and case. Any of the electrodes and the case electrode may be assigned to up to k possible groups or timing “channels.” In one embodiment, by way of example and not limitation, k may equal four. The timing channel identifies which electrodes are selected to synchronously source or sink current to create an electric field in the tissue to be stimulated. Amplitudes and polarities of electrodes on a channel may vary. In particular, the electrodes can be selected to be positive (anode, sourcing current), negative (cathode, sinking current), or off (no current) polarity in any of the k timing channels. The waveform generator may be operated in a mode to deliver electrical modulation energy that is therapeutically effective and causes the patient to perceive delivery of the energy (e.g., therapeutically effective to relieve pain with perceived paresthesia), and may be operated in a sub-perception mode to deliver electrical modulation energy that is therapeutically effective and does not cause the patient to perceive delivery of the energy (e.g., therapeutically effective to relieve pain without perceived paresthesia).

The waveform generator may be configured to individually control the magnitude of electrical current flowing through each of the electrodes. For example, a current generator may be configured to selectively generate individual current-regulated amplitudes from independent current sources for each electrode. In some embodiments, the pulse generator may have voltage regulated outputs. While individually programmable electrode amplitudes are desirable to achieve fine control, a single output source switched across electrodes may also be used, although with less fine control in programming. Neuromodulators may be designed with mixed current and voltage regulated devices.

The neuromodulation system may be configured to modulate spinal target tissue or other neural tissue. The configuration of electrodes used to deliver electrical pulses to the targeted tissue constitutes an electrode configuration, with the electrodes capable of being selectively programmed to act as anodes (positive), cathodes (negative), or left off (zero). In other words, an electrode configuration represents the polarity being positive, negative, or zero. An electrical waveform may be controlled or varied for delivery using electrode configuration(s). The electrical waveforms may be analog or digital signals. In some embodiments, the electrical waveform includes pulses. The pulses may be delivered in a regular, repeating pattern, or may be delivered using complex patterns of pulses that appear to be irregular. Other parameters that may be controlled or varied include the amplitude, pulse width, and rate (or frequency) of the electrical pulses. Each electrode configuration, along with the electrical pulse parameters, can be referred to as a “modulation parameter set.” Each set of modulation parameters, including fractionalized current distribution to the electrodes (as percentage cathodic current, percentage anodic current, or off), may be stored and combined into a modulation program that can then be used to modulate multiple regions within the patient.

The number of electrodes available combined with the ability to generate a variety of complex electrical waveforms (e.g., pulses), presents a huge selection of modulation parameter sets to the clinician or patient. For example, if the neuromodulation system to be programmed has sixteen electrodes, millions of modulation parameter sets may be available for programming into the neuromodulation system. Furthermore, for example SCS systems may have thirty-two electrodes which exponentially increases the number of modulation parameters sets available for programming. To facilitate such selection, the clinician generally programs the modulation parameters sets through a computerized programming system to allow the optimum modulation parameters to be determined based on patient feedback or other means and to subsequently program the desired modulation parameter sets. The modulation device 202 may also be configured to use any one or more of the electrodes within the lead system 207 to sense electrical responses.

FIG. 3 illustrates an embodiment of a programming system such as a programming device 303, which may be implemented as the programming device 103 in the neuromodulation system of FIG. 1. The programming device 303 includes a storage device 308, a programming control circuit 309, and a graphical user interface (GUI) 304. The programming control circuit 309 generates the plurality of modulation parameters that control the delivery of the neuromodulation pulses according to the pattern of the neuromodulation pulses. In various embodiments, the GUI 304 includes any type of presentation device, such as interactive or non-interactive screens, and any type of user input devices that allow the user to program the modulation parameters, such as touchscreen, keyboard, keypad, touchpad, trackball, joystick, and mouse. The storage device 308 may store, among other things, modulation parameters to be programmed into the modulation device. The programming device 303 may transmit the plurality of modulation parameters to the modulation device. In some embodiments, the programming device 303 may transmit power to the modulation device. The programming control circuit 309 may generate the plurality of modulation parameters. In various embodiments, the programming control circuit 309 may check values of the plurality of modulation parameters against safety rules to limit these values within constraints of the safety rules.

In various embodiments, the device(s) may be implemented using a combination of hardware, software and firmware. For example, the circuit of GUI, modulation control circuit, and programming control circuit, including their various embodiments discussed in this document, may be implemented using an application-specific circuit constructed to perform one or more particular functions or a general-purpose circuit programmed to perform such function(s). Such a general-purpose circuit includes, but is not limited to, a microprocessor or a portion thereof, a microcontroller or portions thereof, and a programmable logic circuit or a portion thereof.

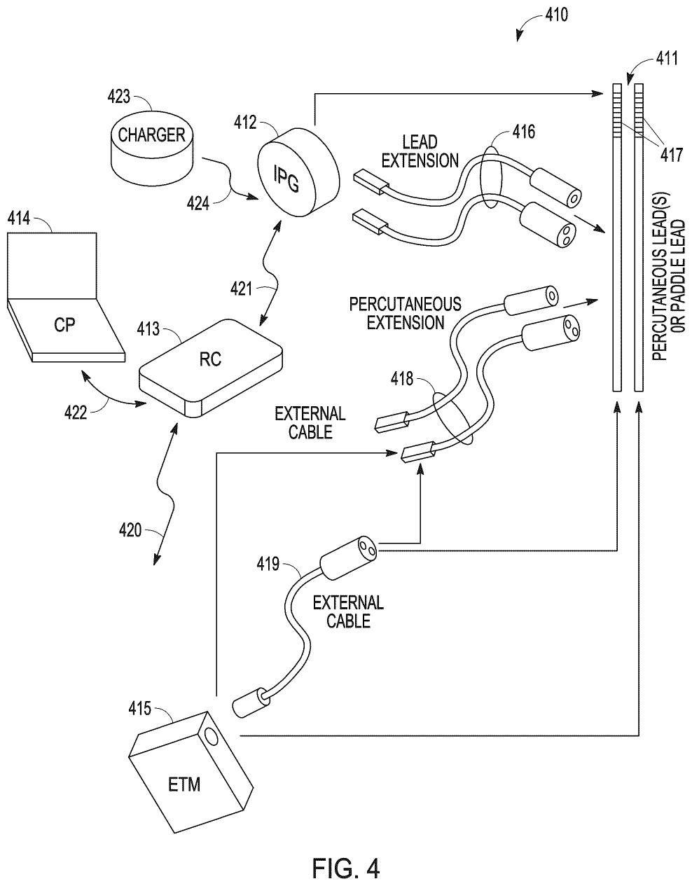

FIG. 4 illustrates, by way of example, an embodiment of a SCS system, which also may be referred to as a Spinal Cord Modulation (SCM) system. The SCS system 410 may generally include one or more (illustrated as two) of implantable neuromodulation leads 411 which also may be paddle lead, an electrical waveform generator 412, an external remote controller (RC) 413, a clinician's programmer (CP) 414, and an external trial modulator (ETM) 415. IPGs are used herein as an example of the electrical waveform generator. However, it is expressly noted that the waveform generator may be configured to deliver regular, repeating patterns of pulses or in complex patterns that appear to be irregular patterns of pulses where pulses have differing amplitudes, pulse widths, pulse intervals, and bursts with differing number of pulses. It is also expressly noted that the waveform generator may be configured to deliver electrical waveforms other than pulses. The waveform generator 412 may be physically connected via one or more percutaneous lead extensions 416 to the neuromodulation lead(s) 411, which carry a plurality of electrodes 417. As illustrated, the neuromodulation leads 411 may be percutaneous leads with the electrodes arranged in-line along the neuromodulation leads. Any suitable number of neuromodulation leads can be provided, including only one, as long as the number of electrodes is greater than two (including the waveform generator case function as a case electrode) to allow for lateral steering of the current. Alternatively, a surgical paddle lead can be used in place of one or more of the percutaneous leads. In some embodiments, the waveform generator 412 may include pulse generation circuitry that delivers electrical modulation energy in the form of a pulsed electrical waveform (i.e., a temporal series of electrical pulses) to the electrodes in accordance with a set of modulation parameters.

The ETM 415 may also be physically connected via the percutaneous lead extensions 418 and external cable 419 to the neuromodulation leads 411. The ETM 415 may have similar waveform generation circuitry as the waveform generator 412 to deliver electrical modulation energy to the electrodes in accordance with a set of modulation parameters. The ETM 415 is a non-implantable device that is used on a trial basis after the neuromodulation leads 411 have been implanted and prior to implantation of the waveform generator 412, to test the responsiveness of the modulation that is to be provided. Functions described herein with respect to the waveform generator 412 can likewise be performed with respect to the ETM 415.

The RC 413 may be used to telemetrically control the ETM 415 via a bi-directional RF communications link 420. The RC 413 may be used to telemetrically control the waveform generator 412 via a bi-directional RF communications link 421. Such control allows the waveform generator 412 to be turned on or off and to be programmed with different modulation parameter sets. The waveform generator 412 may also be operated to modify the programmed modulation parameters to actively control the characteristics of the electrical modulation energy output by the waveform generator 412. A clinician may use the CP 414 to program modulation parameters into the waveform generator 412 and ETM 415 in the operating room and in follow-up sessions. The waveform generator 412 may be implantable. The implantable waveform generator 412 and the ETM 415 may have similar features as discussed with respect to the modulation device 202 described with respect to FIG. 2.

The CP 414 may indirectly communicate with the waveform generator 412 or ETM 415, through the RC 413, via an IR communications link 422 or other link. The CP 414 may directly communicate with the waveform generator 412 or ETM 415 via an RF communications link or other link (not shown). The clinician detailed modulation parameters provided by the CP 414 may also be used to program the RC 413, so that the modulation parameters can be subsequently modified by operation of the RC 413 in a stand-alone mode (i.e., without the assistance of the CP 414). Various devices may function as the CP 414. Such devices may include portable devices such as a lap-top personal computer, mini-computer, personal digital assistant (PDA), tablets, phones, or a remote control (RC) with expanded functionality. Thus, the programming methodologies can be performed by executing software instructions contained within the CP 414. Alternatively, such programming methodologies can be performed using firmware or hardware. In any event, the CP 414 may actively control the characteristics of the electrical modulation generated by the waveform generator 412 to allow the desired parameters to be determined based on patient feedback or other feedback and for subsequently programming the waveform generator 412 with the desired modulation parameters. To allow the user to perform these functions, the CP 518 may include a user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g., CP) may be programmed to provide display screen(s) that allow the clinician to, among other functions, select or enter patient profile information (e.g., name, birth date, patient identification, physician, diagnosis, and address), enter procedure information (e.g., programming/follow-up, implant trial system, implant waveform generator, implant waveform generator and lead(s), replace waveform generator, replace waveform generator and leads, replace or revise leads, explant, etc.), generate a pain map of the patient, define the configuration and orientation of the leads, initiate and control the electrical modulation energy output by the neuromodulation leads, and select and program the IPG with modulation parameters in both a surgical setting and a clinical setting.

An external charger 423 may be a portable device used to transcutaneously charge the waveform generator via a wireless link such as an inductive link 424. Once the waveform generator has been programmed, and its power source has been charged by the external charger or otherwise replenished, the waveform generator may function as programmed without the RC or CP being present.

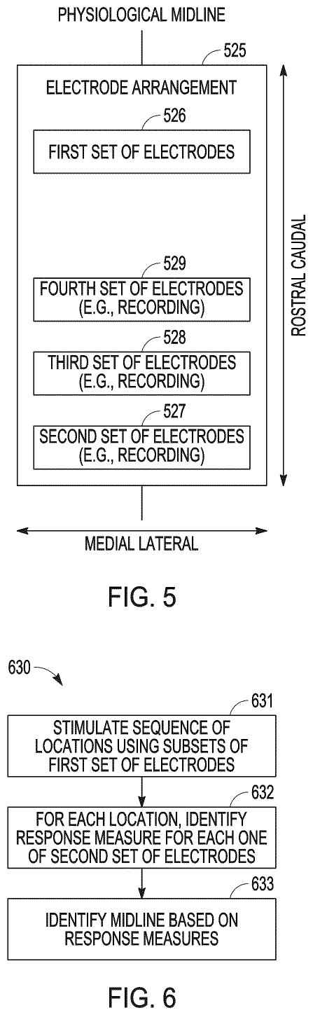

FIG. 5 illustrates, by way of example and not limitation, an example of an electrode arrangement with a stimulating set of electrodes and with recording sets of electrodes. The electrode arrangement 525 may be on a paddle lead or may be on percutaneous leads. The electrode arrangement may be implanted proximate to the spinal cord and may have a length generally in a rostral-caudal direction of the patient and a width generally in a mediolateral direction patient. The electrode arrangement includes sets of electrodes, including at least a first set of electrodes 526 and a second set of electrodes 527 spaced across the width which corresponds to the mediolateral direction across the spinal cord. The first set of electrodes 526 may be used to stimulate locations across the electrode arrangement and the second set of electrodes 527 may be used to record responses to the stimulation using the first set of electrodes 526. The second set of electrodes 527 may be maximally spaced from first set of electrodes 526 in the rostral caudal direction to increase resolution for detecting a relative angle between the electrode arrangement and the spine. However, other embodiments may use a second set of electrodes 527 that are closer to the first set of electrodes 526. Furthermore, some embodiments may use additional sets of electrodes (e.g., third set 528 and fourth set 529) for recording responses to the stimulation using the first set of electrodes. These additional sets of recording electrodes may be used to build plots in a rostrocaudal direction. These plots may be compared against each other which may be used to determine if SCS lead is truly anterior-posterior oriented and parallel respect to the spinal cord.

FIG. 6 illustrates, by way of example and not limitation, a method to identify a midline using responses to a stimulated sequence of locations. The method 630 may be implemented using the electrode arrangement of FIG. 5. As represented at 631, a neurostimulator may be configured to stimulate a sequence of locations of the patient using subsets of the first set of electrodes. Each subset may correspond to a stimulation location. Each subset may include at least two electrodes to provide at least one stimulation electrode and at least one return electrode to stimulate each location. For each stimulated location in the stimulated sequence of locations, the method 630 may include identifying corresponding response measures for each subset within the second set of electrodes 631. For example, the stimulated sequence of locations using the first set of electrodes may include at least a first location and a second location. The second set of electrodes may include at least first electrode and a second electrode. The corresponding response measures may include, when the first location is stimulated, a first first-location response measure for the first electrode and a second first-location response measure for the second electrode. The corresponding response measures may include, when the second location, a first second-location response measure for the first electrode and a second second-location response measure for the second electrode. At 633, the system may be configured to identify a physiological midline based on the corresponding response measures for each stimulated location in the stimulated sequence of locations. The first and second “first-location” response measures may be recorded or measured simultaneously or may be recorded or measured at different times. The first and second “second-location” response measures also may be recorded or measured simultaneously or may be recorded or measured at different times.

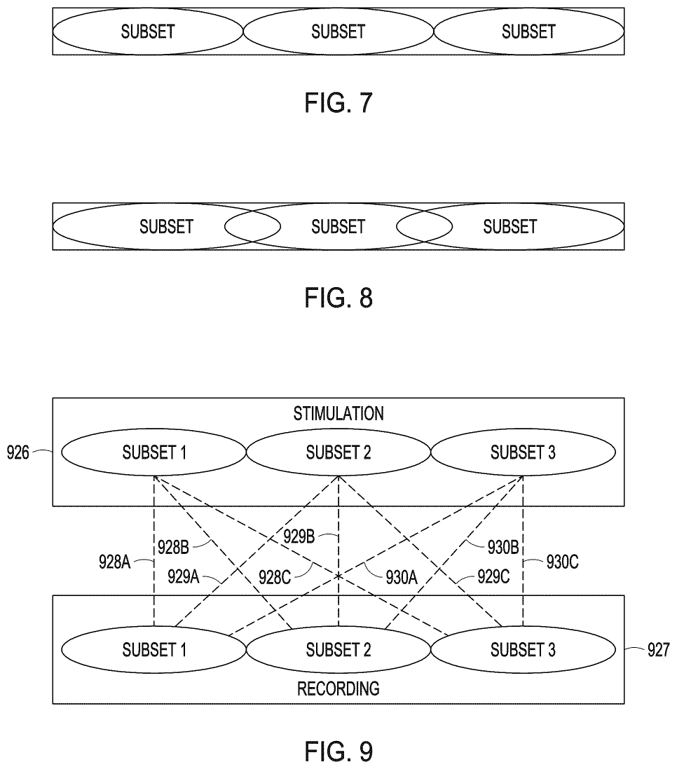

FIG. 7 illustrates, by way of example and not limitation, exclusive subsets of electrodes such as may be used in the stimulating electrodes (e.g., first set 526 in FIG. 5) or recording electrodes (e.g., second set 527 in FIG. 5). For example, subsets of stimulation electrodes may include at least one stimulation electrode and at least one return electrode to stimulate each location. Each subset may include a single electrode (e.g., monopolar stimulation or monopolar sensing). Each subset for may include at least two electrodes. Similarly, subsets of sensing electrodes may include differential pairs. If the subsets are exclusive, an electrode used in one subset is not used in another subset.

FIG. 8 illustrates, by way of example and not limitation, nonexclusive subsets of electrodes such as may be used in the stimulating electrodes (e.g., first set in FIG. 5) or recording electrodes (e.g., second set in FIG. 6). If the subsets are nonexclusive, an electrode used in one subset may be used in another subset as is illustrated by the overlapping subsets. This may be used to provide more stimulation locations and/or sensing locations across the width of the electrode array in the mediolateral direction.

FIG. 9 illustrates, by way of example and not limitation, recording responses using a second set of electrodes 927 when a sequence of locations is stimulated using subsets within a first set of electrodes 926. The sequence of locations may be stimulated using subset 1, then subset 2, and then subset 3 of the first set 926 such that the sequence progressively moves mediolaterally across the spinal cord. However, the locations may be stimulated using the subsets in other orders (e.g., subset 2, then subset 1, and then subset 3). Each stimulated location may create responses, which are recorded at sensing subsets 1, 2, and 3. The recording may be performed simultaneously or may be performed at different times. When a location is stimulated at subset 1, a first “first-location” response measure 928A may be recorded at sensing subset 1, a second “first-location” response measure 928B may be recorded at sensing subset 2, and a third “first-location” response measure 928C may be recorded at sensing subset 3. Similarly, when a location is stimulated subset 2, a first “second-location” response measure 929A may be recorded at sensing subset 1, a second “second-location” response measure 929B may be recorded at sensing subset 2, and a third “second-location” response measure 929C may be recorded at sensing subset 3. When a location is stimulated subset 3, a first “third-location” response measure 930A may be recorded at sensing subset 1, a second “third-location” response measure 931B may be recorded at sensing subset 2, and a third “third-location” response measure 931C may be recorded at sensing subset 3. It is noted that the number of subsets used to stimulate and the number of subsets used to sense may be different, and is further noted that the number of subsets used to stimulate may be different from the number of subsets used to sense.

A curve may be formed by ECAP features for each sensing contact. The mediolateral trend may be used to infer lead position. Trend plots across rows or along individual columns may be used to infer lead alignment with respect to the spinal cord.

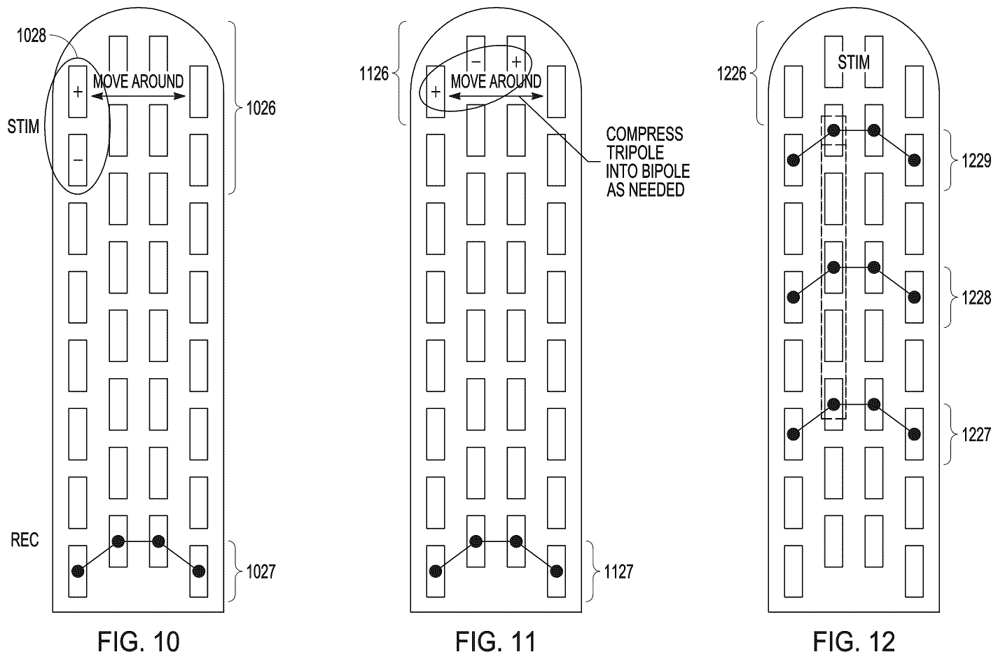

FIG. 10 illustrates, by way of example and not limitation, a paddle lead that shifts a target bipole mediolaterally while recording from other contacts. The electrodes used to provide stimulation may be considered to be a first set of electrodes 1026, and the bipole 1028 formed in each column may be referred to as a subset for the first set of electrodes. The electrodes used to provide the recording may be referred to as a second set of electrodes 1027. The target bipole may shift to move the target pole mediolaterally while recording from relevant contact pairs on the paddle. Recordings may be plotted with respect to mediolateral position to build relative relationships. As stimulation is moved, each of those recording electrode subsets (e.g., monopolar electrode or differential pair) may receive ECAPs or signals that are scaled according to how many dorsal column fibers are activated.

A profile can be developed from the recordings that can inform as to the proximity of the midline and the location of the cathode. For example, when the cathode is centered on midline, the two middle two contacts are likely to sense a bigger signal and the flanking contacts would likely to sense smaller signals that may progress symmetrically. A model, estimates from prior studies, or anatomical knowledge may be used to build an expected profile that's based both on where the cathode is and how far it is expected to be on or off midline. The actual recording profile may be compared to a predicted profile. For example, the predicted profile may be created when the lead is first placed or may be based on an assumption that we're exactly on the midline. By way of example and not limitation, ECAP features that may be used include a magnitude, area under curve (AUC), a curve length, a power spectra properties, wavelet properties. correlation(s) with templates. and a progression with known stimulation parameter (e.g., amplitude, PW).

FIG. 11 illustrates, by way of example and not limitation, a paddle lead that shifts a mediolateral guarded tripole mediolaterally while recording from other contacts. Rather than using a target bipole to stimulate the locations, a mediolateral guarded tripole or a cathodic monopole may be used in an attempt to provide more focused stimulation that selectively engages specific populations of dorsal column fibers. The electrodes used to provide stimulation may be considered to be a first set of electrodes 1126, and the tripole 1129 formed in each column may be referred to as a subset for the first set of electrodes. The tripole may be compressed into a bipole near the edges of the electrode arrangement. The profile for bipole and the profile for a tripole may differ in some ways and we can use multiple target poles to ascertain those differences. The electrodes used to provide the recording may be referred to as a second set of electrodes 1127.

FIG. 12 illustrates, by way of example and not limitation, a paddle lead that builds plots in a rostrocaudal direction. These plots may be compared against each other which may be used to determine if SCS lead is truly anterior-posterior oriented and parallel respect to the spinal cord. For example, instead of recording on one row, three, four or five rows may be used to record. The electrodes used to provide stimulation may be considered to be a first set of electrodes 1226. The electrodes used to provide the recording may be referred to as a second set of electrodes 1227, a third set of electrodes 1228, and a fourth set of electrodes 1229. Not only is the mediolateral orientation evaluated, the system can evaluate how that left to right orientation varies up and down the lead. So, a maximum value may indicate a midline location, but that midline might kind of wobble or shift to the left or right as you move up or down the lead if the lead is crooked for example. The system may be configured to determine if trends for a contact, and/or trends along each row (when compared between rows) are consistent with predicted signal if the lead is truly parallel with dorsal columns. In addition to the ECAP features identified above, latencies between different sensing channels may be used when multiple sets of recording electrodes are used.

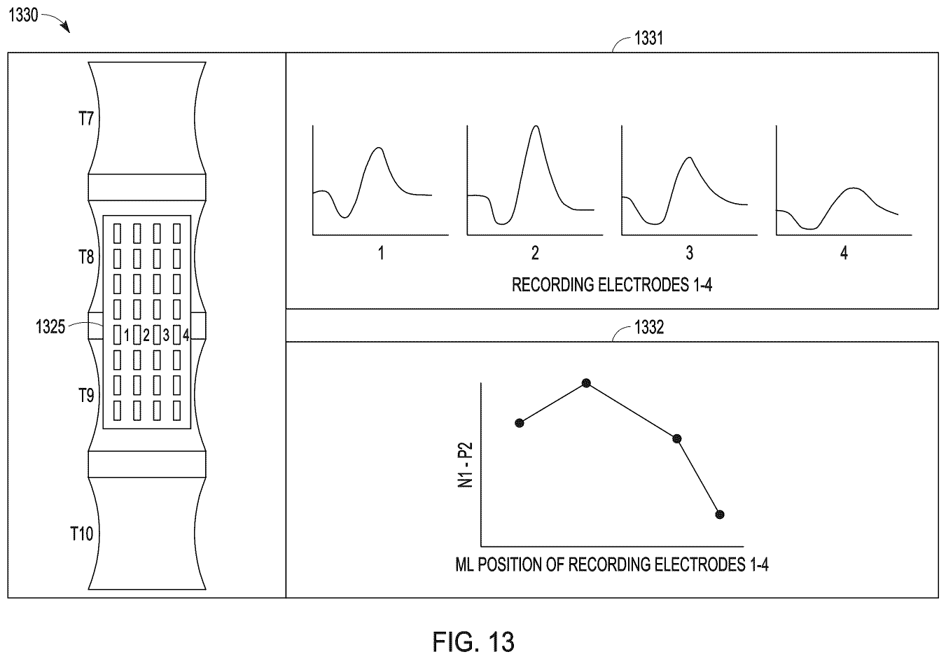

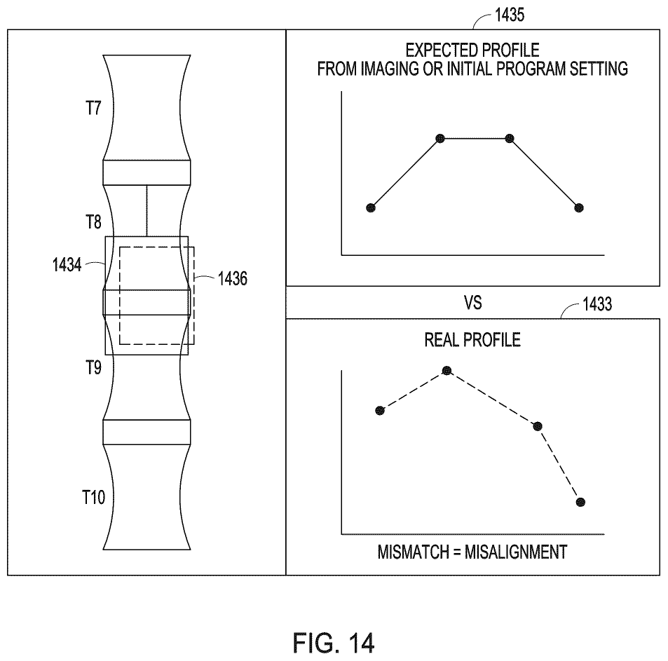

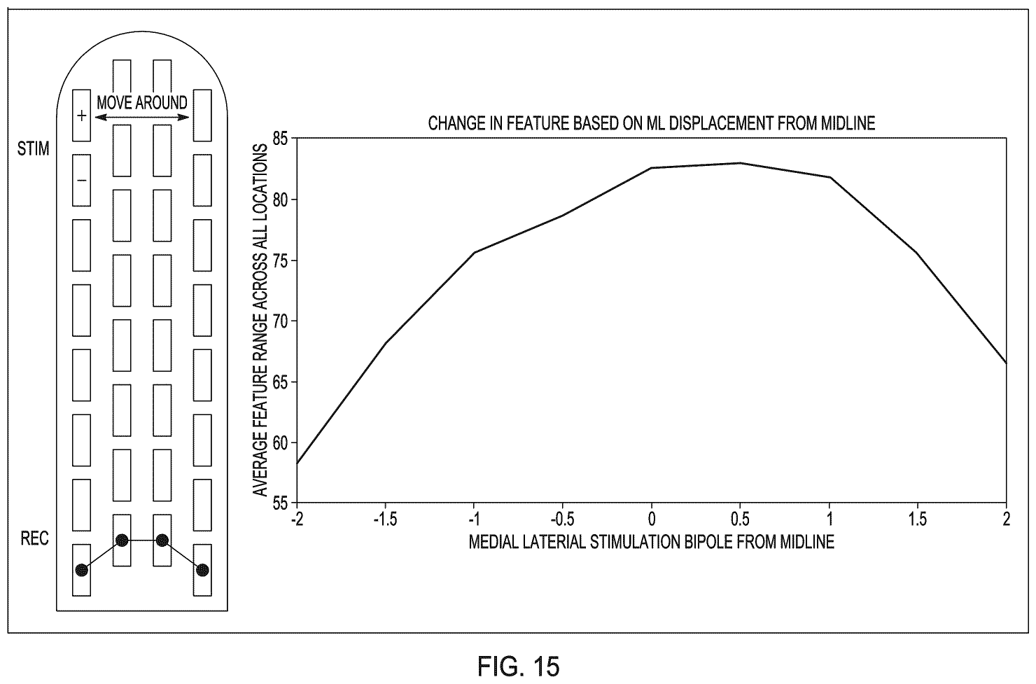

FIG. 13 illustrates, by way of example and not limitation, mediolateral biomarker measurements. An example of a programming screen 1330 for a programmer is illustrated. Medical imaging may be used in providing the image. The figure illustrates an electrode arrangement 1325 over spinal anatomy. Four recording sites are illustrated (“1”, “2”, “3” and “4”). Additionally, a mediolateral stimulation sweep along the top (rostral) of the electrode arrangement 1325 may be performed while recording at each of the recording sites. The sweep may generate recordings as a function of mediolateral (recording) position that could be used to track progression of a recorded biomarker with respect to a recording location. Example recordings at each of these sites are illustrated at 1331, and an ECAP magnitude (e.g., N1-P2) may be plotted for each of these mediolateral recording sites as illustrated at 1332. The highest magnitude is at the second site, which infers that the midline is nearer the second site. The plot in FIG. 13 illustrates a “real profile.” Instead of plotting N1-P2 against the ML position of the recording electrodes, curves may be generated for stimulation positions (e.g., more than four stimulation positions) such as illustrated in FIG. 15.

FIG. 14 illustrates, by way of example and not limitation, a physiological midline identification and recommendation. A characteristic relationship should exist between a biomarker and the mediolateral position. The relationship is expected to be generally symmetric about physiological midline with biomarker (on directly caudal contacts) being the largest where the distal cerebrospinal fluid (dCSF) is thinnest and the most DC fibers are activated. The degree of “imbalance” and/or where an inversion in signal strength with respect to mediolateral position trend occurs may be used as a way to detect and recommend midline, and/or to shift programming accordingly.

An “expected” ratio may be based on user-defined placement, registration from imaging, and the like. A “mismatch” between a model profile (based on initial placement on the programming screen and measured profile) may be usable to “adjust” or recommend an adjustment of the lead position on the programming screen. The profile shape may be monitored and/or maintained within a certain range during periodic calibration checks/if the patient feels an unexpected change. The direction of the shift corresponds to direction of detected imbalance/profile changes.

The system may show how the real profile 1433 compares to an assumed position 1434. The assumed position 1434 may be right on the middle of the spinal cord, resulting in the curve 1435. The real profile 1433 was generated from the actual recordings. These profiles may be compared. The system may be preloaded with a library of profiles and the real profile 1433 may be compared to the library to find the best match and thus find the real placement 1436. The device can then snap to that correct profile, or the one that matches the real profile the best. So, if the paddle is offset, the system may try to find the actual paddle placement that best fits the real profile.

FIG. 15 illustrates, by way of example and not limitation, a change in feature based on mediolateral displacement from midline. The illustration shows a paddle lead with stimulation and recording sets of electrodes, and further shows a plot for an average feature range across all locations against a mediolateral stimulation bi-pole displacement (e.g., in millimeters) from the midline. Sweeping the stimulation mediolaterally relative to the physiological midline of the system causes a change in average evoking potential amplitude across the space sensed with a series of monopoles. A trace (e.g., plot) may be derived by recording on fixed contact(s) all the time, recording on contacts dependent on which contact was used to stimulate, or averaging traces on multiple contacts together. An amplitude or intensity required to produce an evoked neural response of a given size on a given contact may also be plotted against the mediolateral stimulation position.

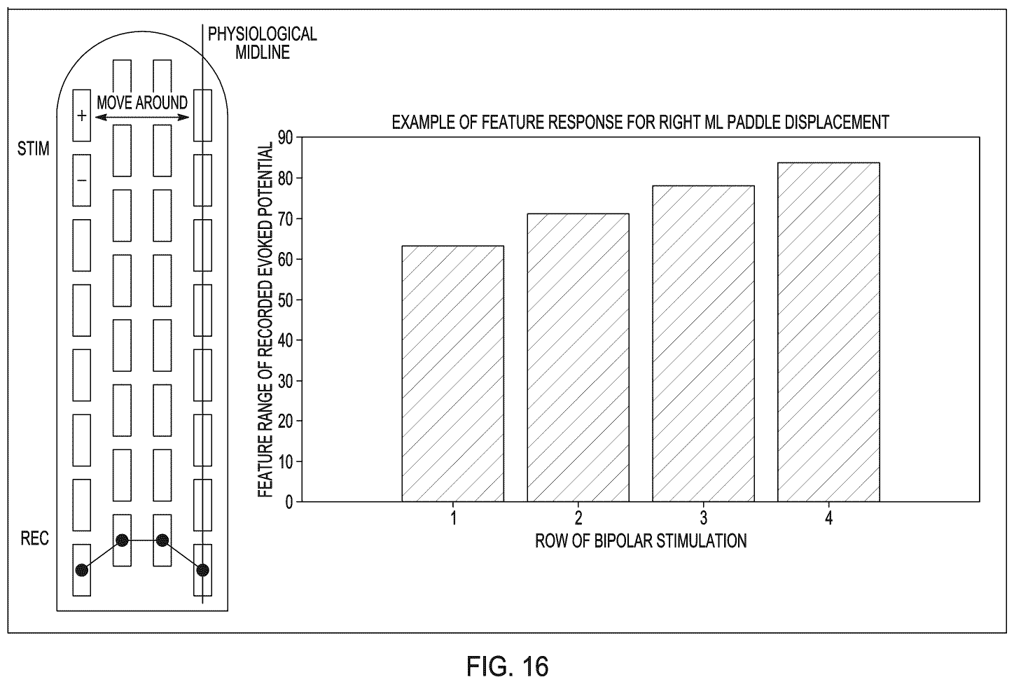

FIG. 16 illustrates, by way of example and not limitation, a feature response for right mediolateral paddle displacement. The midline is shown on a right side the illustrated paddle lead. When the modeled physiological midline is placed off center and the 3 mA stimulation is moved in the medial-lateral direction, the average evoked potential feature range (as sensed by a paddle lead and graphed) is skewed to the direction of the midline. Since the biomarker is expected to be largest when contact is closest to midline when most dorsal columns are activated, the “bias” suggests an imbalance with midline being towards “row” 4. This effect also scales with amplitude as the effect becomes more prominent as you deliver more and more stimulation. The measurements may be repeated for stimulation at different amplitudes to infer the midline with additional certainty.

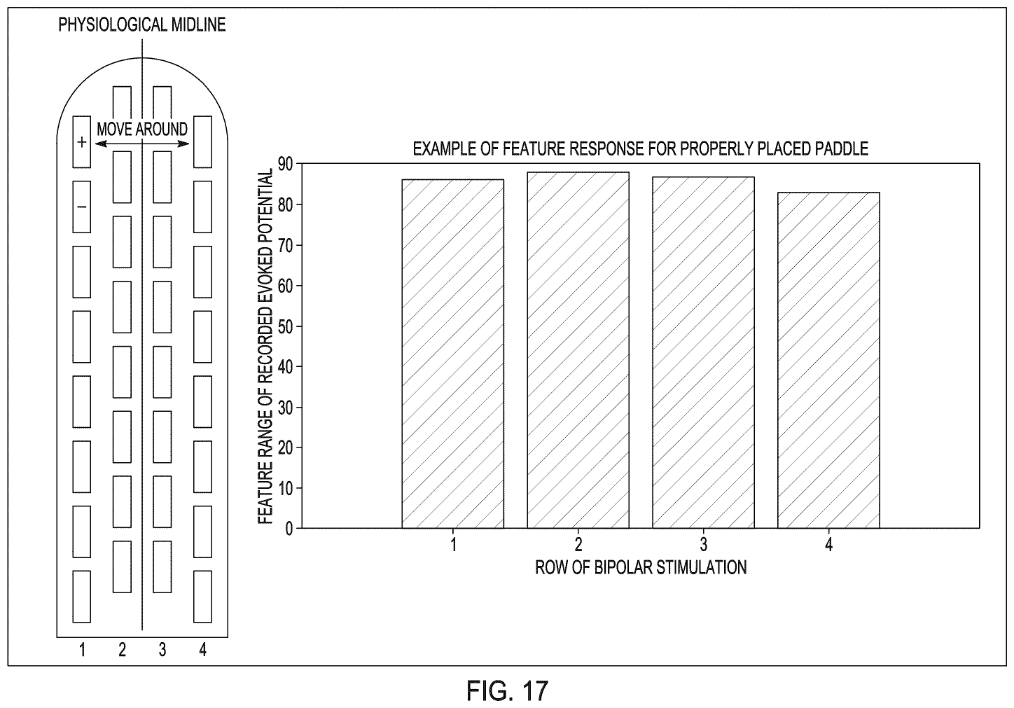

FIG. 17 illustrates, by way of example and not limitation, a feature response for properly placed paddle. When the physiological midline is at or near the center of the lead and when a 3 mA bipolar stimulation is swept across the paddle lead, very little variation is detected as graphed. Since the biomarker is expected to be largest when the contact is closest to midline when most dorsal columns are activated, the symmetry suggests that the midline is between the middle columns.

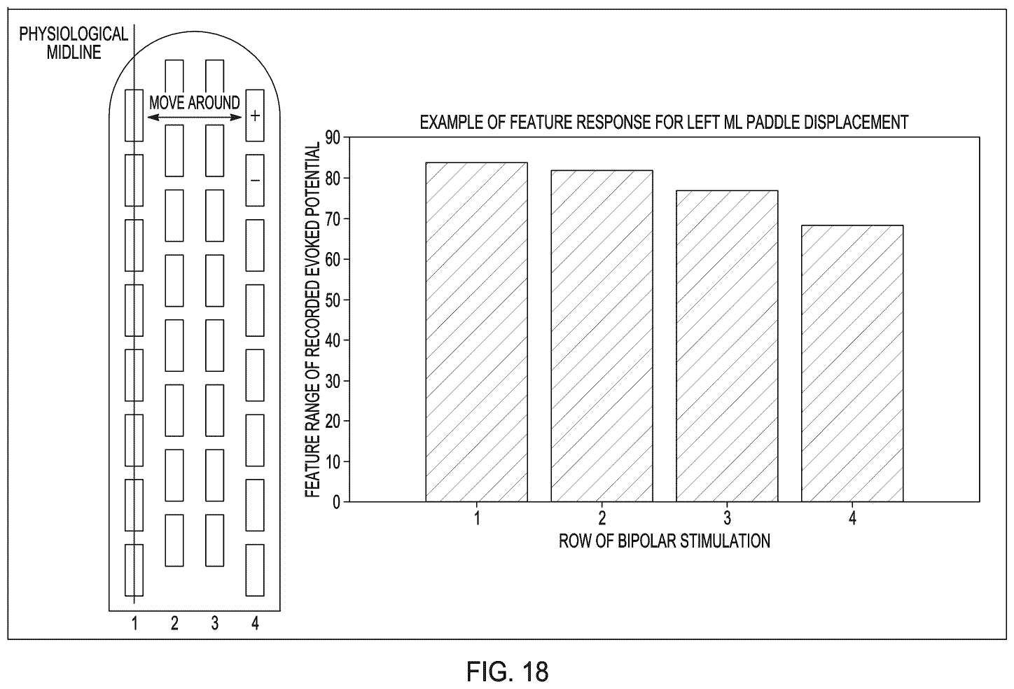

FIG. 18 illustrates, by way of example and not limitation, a feature response for left mediolateral paddle displacement. midline is show on a left side the illustrated paddle lead. When the physiological midline is placed off center and 3 mA stimulation is mediolaterally moved, the average evoked potential feature range (as sensed by a paddle lead) is skewed toward the direction of the midline. Since the biomarker is expected to be largest when the contact is closest to midline when most dorsal columns are activated, the “bias” suggests an imbalance with the midline being towards the first column.

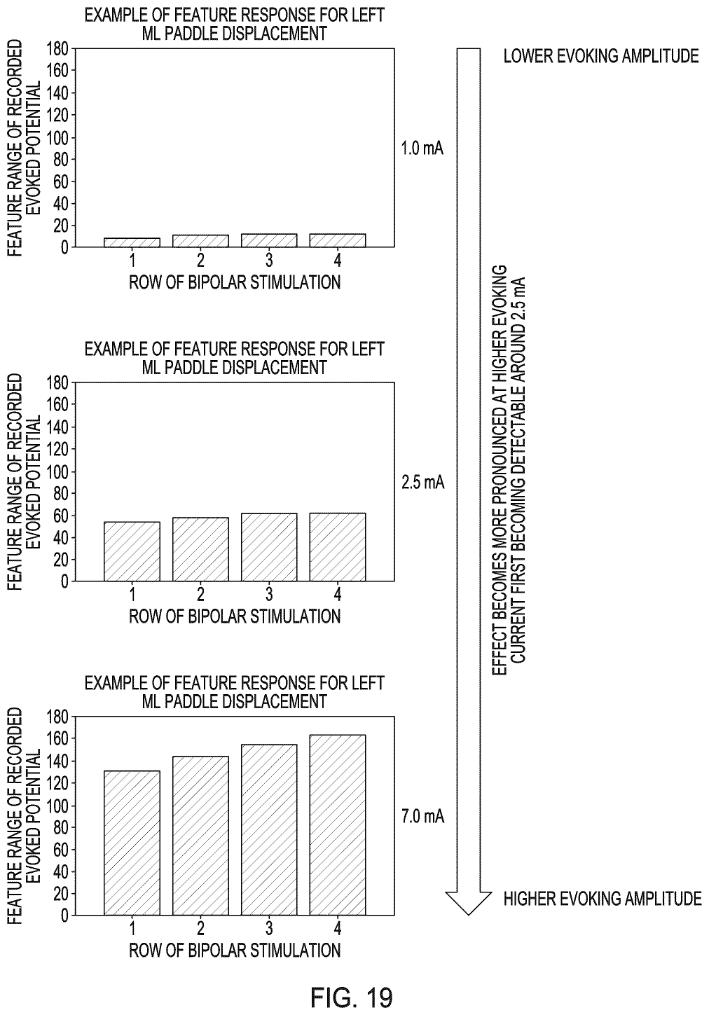

FIG. 19 illustrates, by way of example and not limitation, an effect of increasing amplitude of evoking stimulation. The illustration provides plots with 1.0 mA stimulation, 2.5 mA stimulation and 7.0 mA stimulation. The effect is first detectable around 2.5 mA, and becomes more pronounced at higher stimulation amplitudes. The consistency in trend with a changing amplitude (e.g., increase amplitude) may be used as a confirmatory metric for identifying the midline.

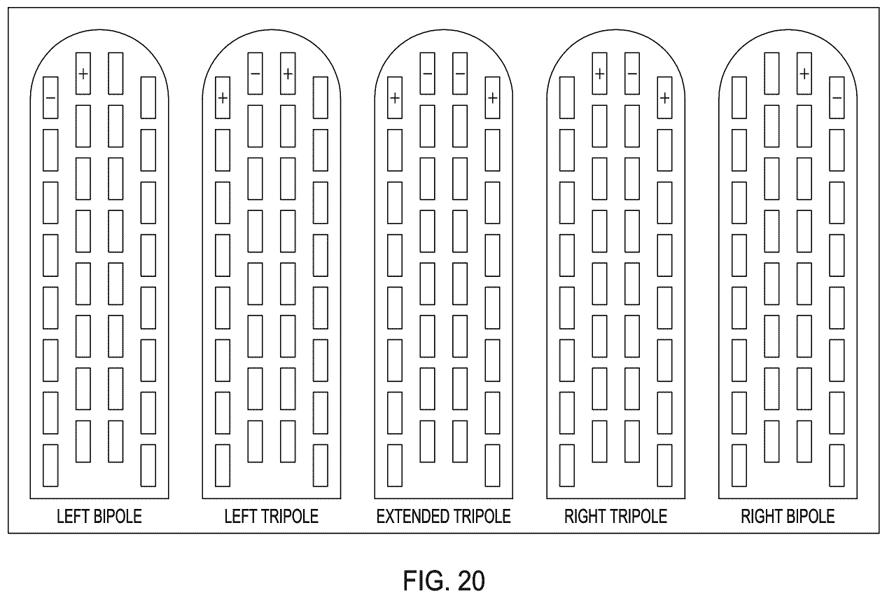

FIG. 20 illustrates, by way of example and not limitation, stimulation configurations. In this non-limiting example used to model the response, the mediolateral stimulation sweep may move from a left bipole, to a left tripole, to an extended tripole, to a right tripole and finally to a right bipole. As identified above, the stimulation may progressively sweep from one side of the electrode arrangement to the other side of the electrode arrangement. Or the stimulation may not be swept, but rather directed to the stimulation locations in a different order such as but not limited to extended tripole, left bipole, left tripole, right bipole, and right tripole.

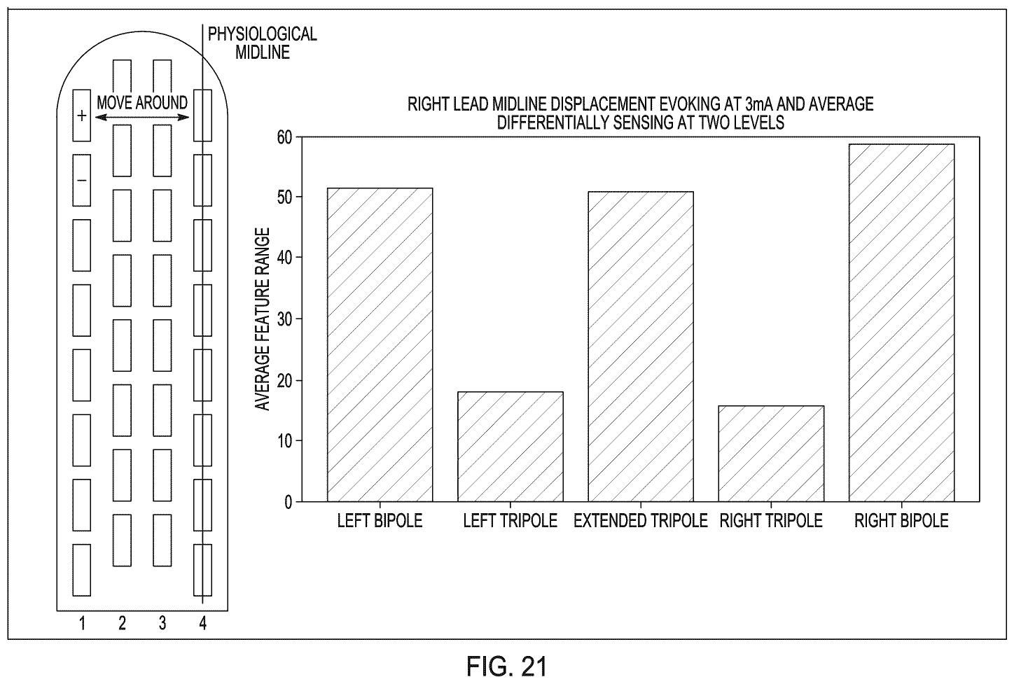

FIG. 21 illustrates, by way of example and not limitation, right lead midline displacement evoking in response to 3 mA stimulation and average differentially sensing at two levels. The right bipole provides the largest signal. The profile will be different because the geometry is different. The way in which the electrodes actually activate the dorsal columns using a tripole is not going to be the same as it was with the bipole. However, it will still generate a characteristic or a distinctive profile that may be used to infer lead placement.

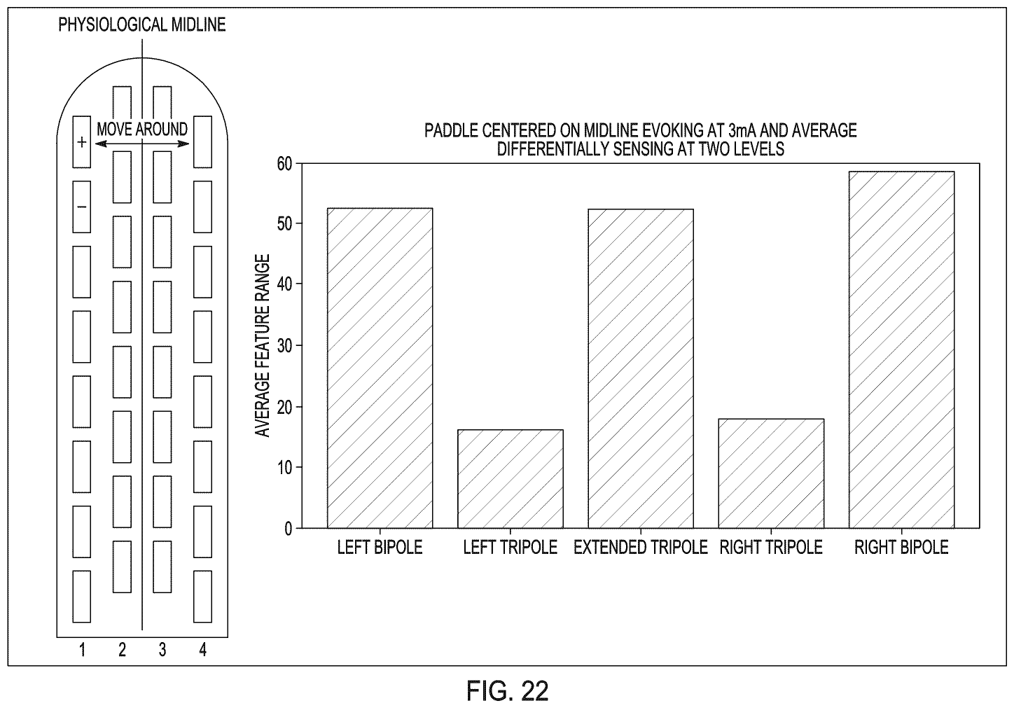

FIG. 22 illustrates, by way of example and not limitation, a paddle center on midline displacement evoking in response to 3 mA stimulation and average differentially sensing at two levels. The plots are relatively symmetric between the left and right bipoles and between the left and right tripoles, which may be used to infer lead placement.

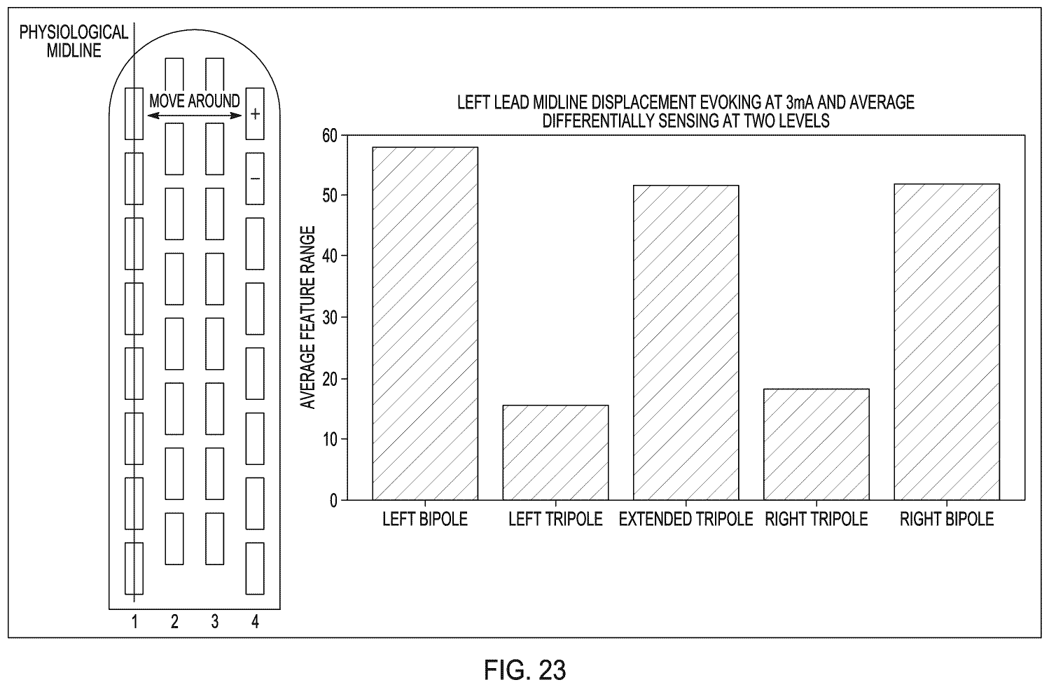

FIG. 23 illustrates, by way of example and not limitation, right lead midline displacement evoking midline displacement evoking in response to 3 mA stimulation and average differentially sensing at two levels. The left bipole is largest, and is larger than the right pole, and the left tripole is larger than the right tripole, which may be used to infer lead placement. Thus, as illustrated in FIGS. 21-23, the response to different geometries may also act as a “profile” that suggests lead placement.

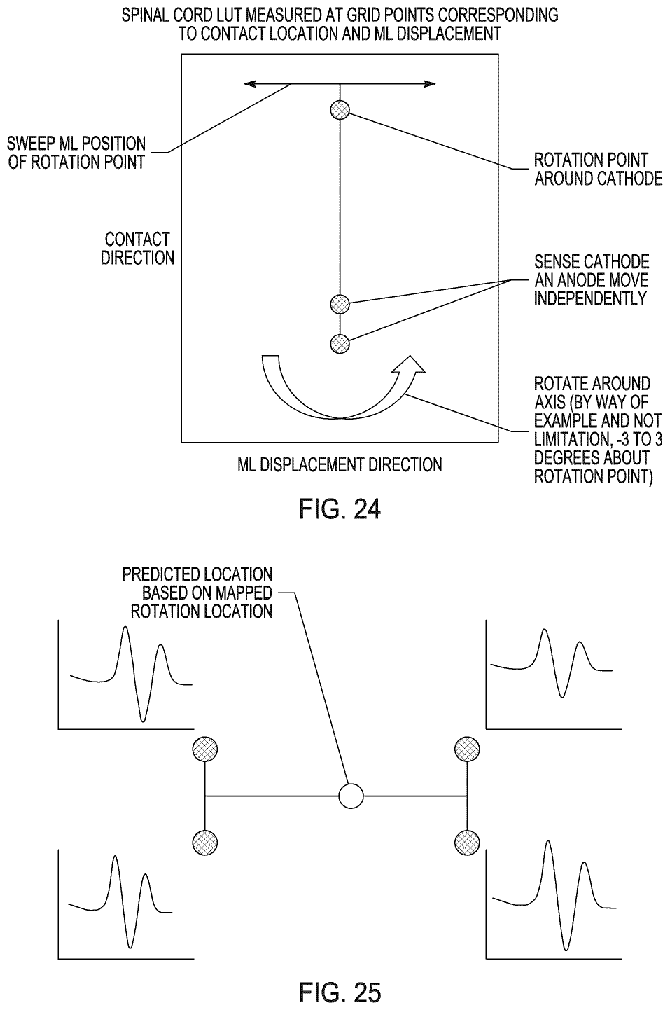

FIG. 24 illustrates, by way of example and not limitation, a simulation of a rotation of a paddle lead. The spinal cord lookup table may correspond to measured grid points corresponding to contact location and mediolateral displacement. The figure illustrates a rotation point around a stimulating electrode (e.g., cathode). A sensing anode and cathode may move independently. The axis may be rotated from −3 to 3 degrees about the rotation point. The mediolateral displacement direction is illustrated by the arrow. The mediolateral position of the rotation point may be swept.

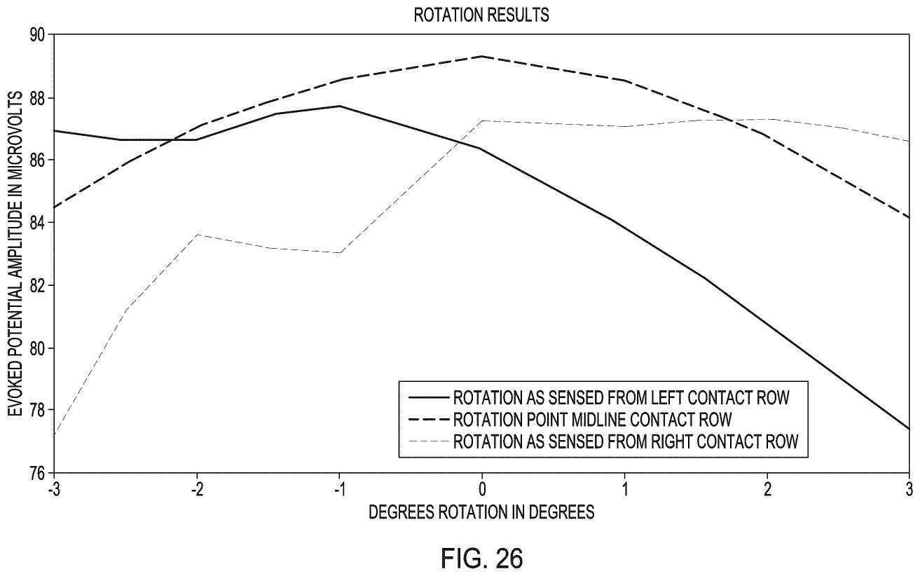

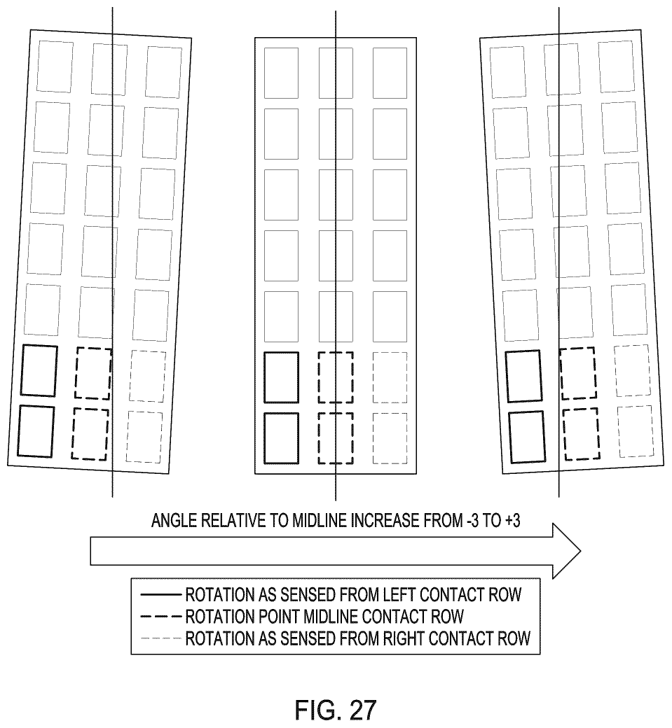

FIG. 25 illustrates, by way of example and not limitation, an interpolation of a sensing contact based on a nearest grid point. The predicted location may be based on a mapped rotation location. An interpolation (e.g., element-wise bilinear interpolation) may be performed on known grid points to estimate the point between the grid points. Some embodiments determine the degree of rotation of the electrode arrangement based on the shape of the recorded action potentials or based on the progression of set action potentials along the lead, such as illustrated in FIGS. 26-27.

FIG. 26 illustrates, by way of example and not limitation, plots that illustrate that rotation of the paddle lead along the mediolateral-rostrocaudal access can be detected using three pairs of contacts on the same paddle lead. FIG. 27 illustrates, by way of example and not limitation, rotation angles of a paddle lead with respect to the midline (e.g., 0 Degrees from Midline, +3 Degrees from Midline, −3 Degrees from Midline), and further identifies the recording electrode differential pairs from which the plots of FIG. 26 were built. The illustrated paddle is a simplification of actual leads to illustrate the determination of rotation. The modeled lead has dimension that include 0.5 mm spacing (center-to-center between columns), 6.6 mm spacing (center-to-center between rows), and sensing using the bottom two rows of contacts, stimulation and sensing from the left contact column, from the middle contact, and from the right contact column. It is noted that the electrodes may be different sizes in different columns. The system may be designed with libraries with entries specific for the paddle that is being used. These figures illustrate that, if you rotate the lead, you can detect rotation as the ECAP gets larger as contacts rotate closer to the midline. Displacement and asymmetry between the flanking differential pairs relative to the middle differential pair becomes more severe as rotation increases. The stimulation may be delivered on a set of contacts assumed to be “midline” or “symmetric” initially. Periodically as calibration checks, the stimulation may be mediolaterally swept to assess symmetry. Steering and lead placement may be adjusted to account for such rotation/displacement.

Preliminary modeling results suggest that the relationship between evoked neural response features and mediolateral position can be used as a way to detect physiological midline, which has the potential to make programming easier. For example, the present subject matter may find the midline without medical imaging, as some known software mirrors the fluoroscopic image to provide drag-and-drop of the lead on the image. And this information may be fed to algorithms/programs that determine the simulation parameters for the implanted lead while accounting for lead offset and relative lead movement during the trial, after the permanent implant, or during the procedure.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A method performed using an electrode arrangement positioned near a spine of a patient, wherein the electrode arrangement has a length generally in a rostral-caudal direction of the patient and a width generally in a mediolateral direction patient, and the electrode arrangement includes a first set of electrodes spaced across the width and a second set of electrodes spaced across the width, wherein the method includes:
stimulating a sequence of locations of the patient using subsets of the first set of electrodes;
for each stimulated location in the stimulated sequence of locations, identifying corresponding response measure for each electrode subset within the second set of electrodes, wherein
the stimulated sequence of locations includes at least a first location and a second location and the second set of electrodes includes at least a first electrode subset and a second electrode subset,
the corresponding response measures includes, for the first location, a first first-location response measure for the first electrode subset and a second first-location response measure for the second electrode subset, and
the corresponding response measures includes, for the second location, a first second-location response measure for the first electrode subset and a second second-location response measure for the second electrode subset; and
identifying a physiological midline based on the corresponding response measures for each stimulated location in the stimulated sequence of locations.

2. The method of claim 1, wherein the stimulating the sequence of locations includes moving at least one pole for delivered stimulation energy to stimulate the sequence of locations.

3. The method of claim 2, wherein the at least one pole includes a bipole, the stimulating the sequence of locations includes moving the bipole for the delivered stimulation energy to stimulate the sequence of locations, and the bipole includes a cathodic pole and an anodic pole.

4. The method of claim 2, wherein the at least one pole includes a mediolateral guarded tripole, the stimulating the sequence of locations includes using the mediolateral guarded tripole to stimulate at least some of the locations, and the mediolateral guarded tripole includes a cathodic pole generally between at least two anodic poles.

5. The method of claim 2, wherein the at least one pole includes a monopole used to stimulate at least some of the locations.

6. The method of claim 2, wherein the electrode arrangement includes at least a third set of electrodes spaced across the width, wherein for each stimulated location in the stimulated sequence of locations, the identifying corresponding response measures includes either:
identifying corresponding response measures for each electrode subset within the third set of electrodes, wherein the system is configured to identify the physiological midline based on the corresponding response measures for each electrode subset within the second set of electrodes and the corresponding response measures for each electrode subset within the third set of electrodes; or
using corresponding electrodes within the second set of electrodes and the third set of electrodes to form corresponding differential pairs for sensing, and use the corresponding differential pairs to identify the corresponding response measures.

7. The method of claim 6, further comprising comparing trend plots across rows for the second set of electrodes and the at least the third set of electrodes to infer lead alignment with the physiological midline.

8. The method of claim 6, further comprising comparing trend plots across individual columns for electrodes in the second and the least the third sets of electrodes.

9. The method of claim 1, wherein the first set of electrodes is proximate a rostral end of the electrode arrangement and the second set of electrodes is proximate to a caudal end of the electrode arrangement.

10. The method of claim 1, wherein the electrode arrangement is on a paddle lead.

11. The method of claim 1, wherein the electrode arrangement includes at least three columns of electrodes.

12. The method of claim 1, wherein the electrode arrangement includes rows of electrodes, and at least some of the electrodes in the rows of electrodes are staggered with respect to each other.

13. The method of claim 1, wherein the corresponding response measures includes measures for at least one feature of an evoked compound action potential (ECAP) selected from a magnitude, an area under curve (AUC), a curve length (CL), a power-spectra property, a wavelet property, a correlation with a template, progression with a known parameter, or at least one latency between different channels.

14. The method of claim 1, further comprising programming a neurostimulator connected to the electrode arrangement to deliver neuromodulation using the identified physiological midline.

15. The method of claim 1, further comprising comparing changes in the corresponding response measures to detect lead migration, and reprogramming a neurostimulator connected to the electrode arrangement based on the detected lead migration.

16. The method of claim 1, further comprising displaying a plot of the corresponding response measures for each of the stimulated locations, wherein the displayed plot is used to identify the physiological midline.

17. A non-transitory machine-readable medium including instructions, which when executed by a machine, cause the machine to perform a method using an electrode arrangement positioned near a spine of a patient, wherein the electrode arrangement has a length generally in a rostral-caudal direction of the patient and a width generally in a mediolateral direction patient, and the electrode arrangement includes a first set of electrodes spaced across the width and a second set of electrodes spaced across the width, wherein the method performed by the machine includes:
stimulating a sequence of locations of the patient using subsets of the first set of electrodes; and
for each stimulated location in the stimulated sequence of locations, identifying corresponding response measures for each electrode subset within the second set of electrodes, wherein
the stimulated sequence of locations includes at least a first location and a second location and the second set of electrodes includes at least a first electrode subset and a second electrode subset,
the corresponding response measures includes, for the first location, a first first-location response measure for the first electrode subset and a second first-location response measure for the second electrode subset, and
the corresponding response measures includes, for the second location, a first second-location response measure for the first electrode subset and a second second-location response measure for the second electrode subset.

18. A system, comprising:
an electrode arrangement configured to be positioned near a spine of a patient, wherein the electrode arrangement has a length generally in a rostral-caudal direction of the patient and a width generally in a mediolateral direction patient, and the electrode arrangement includes a first set of electrodes spaced across the width and a second set of electrodes spaced across the width;
a neurostimulator configured to connect to the electrode arrangement and further configured to:
stimulate a sequence of locations of the patient using subsets of the first set of electrodes;
for each stimulated location in the stimulated sequence of locations, identify corresponding response measures for each electrode subset within the second set of electrodes, wherein
the stimulated sequence of locations includes at least a first location and a second location and the second set of electrodes includes at least a first electrode subset and a second electrode subset,
the corresponding response measures include, for the first location, a first first-location response measure for the first electrode subset and a second first-location response measure for the second electrode subset, and
the corresponding response measures include, for the second location, a first second-location response measure for the first electrode subset and a second second-location response measure for the second electrode subset,
wherein the system is configured to identify a physiological midline based on the corresponding response measures for each stimulated location in the stimulated sequence of locations.

19. The system of claim 18, wherein:
the neurostimulator is configured to identify the physiological midline; or
the system includes a programmer configured to program the neurostimulator and the programmer is configured to identify the physiological midline.

20. The system of claim 18, wherein the corresponding response measures includes measures for at least one feature of an evoked compound action potential (ECAP) selected from a magnitude, an area under curve (AUC), a curve length (CL), a power-spectra property, a wavelet property, a correlation with a template, progression with a known parameter, or at least one latency between different channels.
